(19) United States
(12) Patent Application Publication
(10) Pub. No.: US 2018/0000881 A1
(43) Pub. Date: Jan. 4, 2018

(54) Phosphatidylserine-Containing Diet Supplement Aids With Melatonin Anti-Catabolic Dietary Nutrients

(71) Applicant: Bob Fritz, Martinez, CA (US)

(72) Inventor: Bob Fritz, Martinez, CA (US)

(21) Appl. No.: 15/545,567

(22) PCT Filed: Jan. 22, 2016
(86) PCT No.: PCT/US2016/014549

§ 371 Date: Jul. 21, 2017

Related U.S. Application Data
(60) Provisional application No. 62/106,355, filed on Jan. 22, 2015.

(51) Int. Cl. A61K 36/736; A23L 33/10; A61K 36/28; A61K 33/06; A61K 31/685; A61K 9/48; A61K 9/20; A61K 9/14; A61K 9/08; A61K 9/00; A61K 36/286; A23L 33/00; A23L 33/16; A23L 33/105; A61K 36/48

(52) U.S. Cl. CPC A61K 36/736; A61K 36/48; A23L 33/10; A61K 36/28; A61K 33/06; A61K 31/685; A61K 9/48; A61K 9/20; A61K 9/14; A61K 9/08; A61K 9/006; A61K 9/0056; A61K 9/0053; A61K 9/0014; A61K 36/286; A23L 33/40; A23L 33/16; A23L 33/105; A23L 33/30; A23V 2002/00

(57) ABSTRACT

A supplement includes plant-derived phosphatidylserine in a physiologically beneficial amount and at least one plant-based concentrate or extract which promotes a melatonergic benefit.

### RELATED APPLICATION

This application claims the benefit of U.S. Provisional Application No. 62/106,355, filed on Jan. 22, 2015. The entire teachings of the above application are incorporated herein by reference.

### BACKGROUND

Sleep plays an important role in overall physical health and wellbeing. Weight gain, high blood pressure, decreased memory, retinal damage and decreased immunity are among the ill effects that can be experienced by those with chronic sleep loss. For most living beings, including humans, a daily cycle that includes periods of wakefulness and sleep follows a twenty-four hour rhythm, referred to as the circadian rhythm. Americans don't get enough sleep. While people spend about 7.5 hours a night in bed, they only sleep an average of 6.1 hours. The problem has become progressively worse during the past century. In 1900, people slept approximately nine hours a night. That declined to seven hours a night in the 1970s and has decreased further today.

Inadequate sleep is linked to poor concentration, decreased learning ability, reduced attention to detail, and motor vehicle accidents. Worse, poor sleep is ruining our health, causing problems such as obesity, diabetes, high blood pressure, cardiovascular disease, and premature aging.

Chronic sleep deprivation triggers an inflammatory response similar to stress. Sleep loss causes an increase in the stress hormone cortisol. This is important during a single bout of stress because cortisol gives us an energy boost that helps us cope with dangerous situations. Chronically elevated cortisol levels, such as from regular sleep deprivation, promotes chronic inflammation, which takes a terrible toll on health. Chronic inflammation is linked to heart disease, stroke, diabetes, obesity, and immune system suppression.

Sleep is critical for protein synthesis. Lack of sleep causes tissue breakdown marked by increases in catabolic hormones such as cortisol and decreases in anabolic hormones such as testosterone and insulin-like growth factor one (IGF-1).

Inadequate sleep slows pathways involved in protein synthesis, increases protein breakdown, and promotes loss of muscle mass and muscle degradation. In athletes, inadequate sleep slows injury repair and tissue recovery. In older adults, sleep deprivation can contribute to sarcopenia, the loss of muscle tissue.

Exposure to artificial light, and particularly blue light, is known to interfere with circadian rhythms such as by suppressing the secretion of melatonin in the body, thus preventing the body from entering a natural period of sleep. Additionally, elevated cortisol levels, resulting from exposure to artificial light or experienced during times of physical and/or mental stress, can significantly disrupt natural sleep patterns. Blue light increases the activity of an enzyme called indoleamine 2,3-dioxygenase 1 (IDO), which is linked to melatonin breakdown and sleep impairment. IDO converts tryptophan, an important chemical for sleep, into kynurenine. Metabolites of kynurenine have toxic effects on brain function that include cell death and atrophy of the hippocampus, a brain center involved in memory and psychological orientation.

Inadequate sleep also increases the production of chemicals called reactive oxygen species (ROS) or free radicals. These are highly reactive chemicals that trigger inflammation, cell destruction, DNA damage, and immune system depression. ROS contribute to the grogginess and low energy levels associated with inadequate sleep.

Backlit electronic devcies, e.g., laptop computers, televisions, tablets and smart phones suppress melatonin, increase the destructive chemicals IDO and cortisol, and promote the production of ROS—all of which are disruptive to sleep.

Insufficient sleep has been recognized as a severe public health problem by the Centers for Disease Control (CDC), with an estimated 50-70 million adults in the United States having some form a sleep or wakefulness disorder. The cost to employers exceeds $63 billion annually. Accordingly, there is a need for compositions and methods that can promote healthful sleep cycles.

### SUMMARY OF THE INVENTION

The invention relates in part to certain compositions that can be taken as a supplement to a healthful diet, and methods of use and administration thereof.

For example, a dietary supplement can contain plant-based components that blunt cortisol and exhibit melatonergic benefits. The dietary supplement can be utilized as a sleep aid, counteracting the effects of blue light exposure. The dietary supplement can additionally counteract the effects of excess cortisol and, further, can provide anti-inflammatory benefits and strengthen immunity.

In one embodiment, a supplement includes phosphatidylserine and a tart cherry component. The phosphatidylserine can be a plant-derived phosphatidylserine, such as soy-derived phosphatidylserine (“SDPS”). The phosphatidylserine can constitute between about 10% and about 35% by mass of the supplement. The supplement can include about 30 mg to about 130 mg of SDPS. The tart cherry component can be any one of dried tart cherry, tart cherry powder, and tart cherry extract. The tart cherry component can constitute between about 10% and about 70% by mass of the supplement. The supplement can include about 30 mg to about 250 mg of tart cherry.

The supplement can further include divalent magnesium, a feverfew component, and/or a safflower seed component. The divalent magnesium can be supplied as an orotate salt and can constitute between about 20% and about 60% by mass of the supplement. The supplement can include about 70 mg to about 250 mg of divalent magnesium. The feverfew component can constitute between about 5% and about 20% by mass of the supplement. The supplement can include about 15 mg to about 100 mg of the feverfew component. The safflower seed component can constitute between about 1% and about 30% by mass of the supplement. The supplement can include about 3 mg to about 110 mg of the safflower seed component. The supplement can further include an encapsulant, such as an encapsulant of chitosan, cyclodextrin, cyclodextrin dendrimer, lecithin, and a plant protein

In another embodiment, a diet supplement includes plant-derived phosphatidylserine in a physiologically beneficial amount and at least one plant-based concentrate or extract which promotes a melatonergic benefit. A physiologically beneficial amount of phosphatidylserine can be of about 30 mg to about 130 mg. The at least one plant-based concentrate or extract can be of prunus cerasus (tart cherry), tanacetum parthenium (feverfew), and/or carthamus tinctorius (safflower). The supplement can include about 30 mg to about 250 mg of prunus cerasus, about 15 mg to about 100 mg of tanacetum parthenium, and/or about 3 mg to about 110 mg of carthamus tinctorius.

The phosphatidylserine and the at least one plant-based concentrate or extract can be contained within a capsule, a tablet, a liquid (for oral administration), an edible par, a powder, a topical medicament, and/or a sublingual tablet or strip.

In one embodiment, a composition includes about 50% to about 70% of a magnesium component, about 10% to about 30% soy-derived phosphatidylserine (SDPS), about 5% to about 20% of a tart cherry component, about 5% to about 15% of a feverfew component, and about 1% to about 5% of a safflower seed component.

In another embodiment, a composition includes about 150 mg to about 250 mg of a magnesium component, about 50 mg to about 100 mg soy-derived phosphatidylserine (SDPS), about 25 mg to about 45 mg of a tart cherry component, about 25 mg to about 45 mg of a feverfew component, and about 1 mg to about 5 mg of a safflower seed component.

In one embodiment, a method of treating a condition in a subject includes administering a supplement including phosphatidylserine and a tart cherry component to the subject. In another embodiment, a method of treating a condition in a subject includes administering a supplement including plant-derived phosphatidylserine in a physiologically beneficial amount and at least one plant-based concentrate or extract which promotes a melatonergic benefit. The condition can be any one of jet lag, depression, weight loss, mood disorder, dysthymia, anxiety, seasonal affective disorder, sleep disorder, circadian rhythm disorder, light-induced sleep disruption, or a combination thereof.

In another embodiment, a method of providing a healthful composition to a subject includes administering a supplement including phosphatidylserine and a tart cherry component to the subject. In another embodiment, a method of providing a healthful composition to a subject includes administering a supplement including plant-derived phosphatidylserine in a physiologically beneficial amount and at least one plant-based concentrate or extract which promotes a melatonergic benefit. The healthful composition can be provided during at least one of physical training and dieting.

In another embodiment, a method of promoting wellbeing in a subject includes providing and administering to the subject a composition including phosphatidylserine and a tart cherry component, and/or a composition including plant-derived phosphatidylserine in a physiologically beneficial amount and at least one plant-based concentrate or extract which promotes a melatonergic benefit. The wellbeing being promoted can be wellbeing of at least one of eye health and digestion.

In another embodiment, a method of regulating activity of at least one of tryptophan 1,3-dioxygenase (TDO) and indoleamine 2,3-dioxygenase 1 (IDO) in a subject, includes administering to the subject a composition including phosphatidylserine and a tart cherry component, and/or a composition including plant-derived phosphatidylserine in a physiologically beneficial amount and at least one plant-based concentrate or extract which promotes a melatonergic benefit.

### DETAILED DESCRIPTION OF THE INVENTION

A description of example embodiments of the invention follows.

Cortisol, Melatonin, and Health

Cortisol is a hormone that has a variety of effects, including in the body's circadian rhythms. Levels of cortisol typically dip around an individual's bedtime and increase over the night hours. Cortisol levels peak in the morning, helping to promote alertness upon waking, and gradually decline over the day. During stressful times, elevated levels of cortisol are known to cause sleep disturbances, including insomnia, frequent wakening, fragmented sleep, and decreased amounts of slow-wave sleep.

Cortisol production is also known to be controlled, at least in part, through exposure to light. The Suprachiasmatic Nucleus (SCN) is a group of cells located in the hypothalamus that responds to light and dark signals received by the eyes. Upon the detection of light, the SCN signals the production of hormones, including cortisol, to assist with wakefulness. Elevated cortisol is known to stimulate tryptophan 1,3-dioxygenase (TDO) levels, associated with depression and lowered immunity.

Melatonin is another hormone that plays a role in the body's circadian rhythms. Melatonin is produced in the tryptophan/serotonin pathway and is actively secreted by the adrenal cortex in the evening hours to induce sleep. The SCN signals the production of melatonin upon the detection of darkness. Extended periods of light can cause the SCN to delay signaling for the release of melatonin.

Effects of Ambient Light on Health

Sleep/wake cycles are regulated in part by exposure to ambient light. The body is particularly sensitive to short-wavelength light (e.g., within the blue-light spectrum of about 450-495 nm). However, both short-wavelength and longer-wavelength light (e.g., light within the red-light spectrum of about 620-750 nm) can induce alertness at night. The phenomenon of blue light exposure and its interference with sleep has been increasing in recent times, owing to the widespread use of digital screens.

With growing sources of ambient light, e.g., light pollution from indoor and outdoor sources, the sleep/wake cycle can become disrupted. The use of electronic devices with light display is one source of light, and can have an effect on the SCN and the signaling of sleep. The use of electronic light displays can also have an effect on wake-inducing hormones, such as melatonin and cortisol. Exposure to the light generated by electronic devices, for example, televisions, computer screens, mobile phone screens, tablet screens, and the like, can cause the SCN to delay the release of melatonin, extend the release of cortisol, or both. Even small electronic devices can emit light in amounts that are sufficient to miscue the brain and promote wakefulness. Even sleeping subjects with eyes closed are affected by blue light, e.g. via melatonin catabolism. This can be particularly true of devices that are intended to be held and viewed relatively close to the eyes, e.g., computers, tablets, smart phones, and the like. While electronic devices can emit light of all, or a number of, colors, they can still emit a significant amount of blue light, which can particularly interfere with the body's sleep-signaling pathways.

Healthful Compositions

In some embodiments, the invention relates to a composition that regulates aspects of an individual's (e.g., human, primate, mammal) production, metabolism, and/or elimination of cortisol, melatonin, or both. In some embodiments, the composition blocks, attenuates, or reduces an effect of light (e.g., blue light) on a subject (e.g., by blocking pathways that can lead to reduced production or secretion of melatonin or increased catabolism of melatonin in response to light exposure). In some embodiments, the composition improves the duration and/or quality of sleep and/or regulates the sleep/wake cycle.

For example, in some embodiments, a composition can include one or any combination of a cortisol reducing agent and a melatonergic agent.

In some embodiments, and without wishing to be bound by any particular theory or mechanism, a composition can include one or any combination of the following: phosphatidylserine (“PS”), e.g, soy-derived phosphatidylserine (“SDPS”), prunus cerasus (“tart cherry”), tanacetum parthenium (“feverfew”), hypericum (“St. John's Wort”), carthamus tinctorius (“safflower”) (e.g., safflower seed) and magnesium salt (magnesium”).

### Cortisol Reducing Agents

A cortisol reducing agent is a chemical or compound that can block the effect(s) of cortisol, reduce its production or presence in the body, or both. For example, a cortisol reducing agent, or cortisol blunting agent, can reduce cortisol levels (either average levels or levels at a particular time or day or part of the circadian cycle, e.g., nighttime, REM sleep, pre-wakefulness, during dark/diminished light conditions, post-waking) in a relevant physiological compartment (e.g., blood, brain, others) by, for example up to about 30%, 50%, or 75%. For example, a cortisol reducing agent can reduce cortisol levels by 5%, 10%, 25%, 30%, or 31%.

Examples of Agents. Cortisol reducing agents include phosphatidylserine (“PS”) (e.g., soy-derived phosphatidylserine, SDPS). SDPS is a natural substance that is safe and beneficial for suppressing the production of elevated levels of cortisol. SDPS has been used in conjunction with dietary and fitness programs to inhibit muscle loss or promote muscle gain, as described in U.S. Pat. No. 6,673,378, the entire contents of which are incorporated herein by reference. SDPS is also used as a supplement to promote muscle retention and development in diet and exercise programs where induced stress generates elevated levels of protein consuming glucocorticoids, in particular cortisol. Naturally occurring in plants, phosphatidylserine can be extracted in commercial quantities from soybeans, e.g., as a byproduct of soy lecithin. PS, e.g, SDPS, is one of a variety of substances that have been identified as cortisol antagonists.

Other examples of cortisol-reducing agents include anabolic steroids. Anabolic steroids, however, can have serious side effects and should be administered with care.

Mineral sources of cortisol reducing agents include magnesium (magnesium salt), which can assist with the regulation of cortisol production. Magnesium is a known sleep-promoter, and magnesium deficiencies have been correlated with high cortisol levels. As magnesium can induce a laxative effect when ingested, in a preferred embodiment, compositions including magnesium include magnesium orotate and/or magnesium citrate. Magnesium citrate and magnesium orotate are more easily digested forms of magnesium that can produce less of a laxative effect than other forms of magnesium.

Melatonergic Agents

A melatonergic agent is a chemical or compound that can modulate the production or presence of melatonin in the body. For example, a melatonergic agent can enhance melatonin levels (either average levels or levels at a particular time of day or part of the circadian cycle, e.g., nighttime, REM sleep, pre-wakefulness, during dark/diminished light conditions, post-waking) in a relevant physiological compartment (e.g., blood, brain, others) by at least 1%, 2%, 5%, 10%, 25%, 50%, or 99%.

Melatonin levels can be measured in a variety of ways, including by measuring levels in urine, blood plasma, or saliva (or levels of a metabolite, e.g., 6-sulphatoxymelatonin (aMT6s)). Changes in melatonin levels can be expressed, for example, as a percent change from a baseline, e.g., a changes in 24-hour concentrations relative to pre-treatment baselines, changes in maximum level relative to baseline, changes in average or maximum levels at a specific time in the circadian cycle relative to baseline (e.g., at approximately 8 AM, 10 AM, noon, 2 PM, 4 PM, 6 PM, 8 PM, 10 PM, midnight, 2 AM, 4 AM or 6 AM of actual time or subjective circadian time). Similar methods can be used to measure changes in cortisol after administration of a composition described herein, including via analysis of serum, saliva, hair, urine. Measurement of metabolites can also be used.

Melatonergic agents can include melatonin sources and/or enzyme inhibitors, which have a bearing on the production of melatonin, or its precursors, in the body.

Examples of plant-based melatonin sources include a variety of plants, such as tart cherry.

Examples of enzyme inhibitors include inhibitors of tryptophan 1,3-dioxygenase (TDO) and indoleamine 2,3-dioxygenase (IDO), two enzymes which regulate systemic tryptophan levels, tryptophan being a precursor to melatonin. Light exposure can affect enzymatic activity involved in the metabolism of tryptophan. Tryptophan catabolism results in toxic byproducts and kynurenine production. In some embodiments, enzyme inhibitors blunt the effect of light exposure on the enzymatic-mediated catabolism of tryptophan, melatonin, and/or precursors.

Plant-based melatonergic agents that can function as enzyme inhibitors include tart cherry and safflower seeds.

Further, melatonergic agents include, for example, adaptogens, which are natural substances that can exert a normalizing effect on bodily processes. Adaptogens include, for example, ginseng, holy basil, ashwaganda, astragalus root, licorice root, rhodiola.

### Sources of Agents

Melatonergic agents and cortisol-reducing agents can be, or can be obtained from, a wide variety of sources. For example, they can be plant-based, animal-based, algae-based, synthetic, or semi-synthetic. Plant-based or semi-synthetic melatonergic agents can be extracted or processed from a natural source, such as an herb, fruit, vegetable, plant, root, nut, or, seed. An extract is a solution or preparation containing ingredients or essence of a substance in a concentrated form.

As used herein, a component of a substance (e.g., a plant) is the substance itself or a part, or constituent thereof, and it can be supplied in a variety of forms, e.g., as an extract, oil, powder, concentrate, essential oil, gum, tar, distillate, extract (e.g., into water, alcohol (ethanol)), expressate, and/or liquefaction of the substance or component.

Extracts can be obtained by pressure, distillation, treatment with solvents, or the like. In the alternative, the agent can be administered as the natural material (e.g., leaf, seed, root) in a matrix that contains additional natural, e.g., plant material. As another example, the agent can be a component of an unprocessed or processed (e.g., dried, pulverized, juiced) herb, fruit, vegetable, plant, root, nut, or, seed. Any of the plant-based agents described can be organic or organically grown, such that they were not exposed to one or more of any artificial or synthetic pesticides, herbicides, acaricides, or fungicides. Additionally, any of the plant-based agents can be sustainably grown and/or harvested.

### Embodiments

In some embodiments, and without wishing to be bound by any particular theory, further embodiments of the invention relate to compositions including one or any combination of the following substances and/or components thereof:

phosphatidylserine (“PS”), e.g., soy-derived phosphatidylserine (“SDPS”) in amounts of about 10 mg to about 400 mg (e.g., 9.5 mg, 10 mg, 35, mg, 52.5 mg, 70 mg, 90 mg, 122.5 mg, 105 mg, 140 mg, 210 mg 245 mg, 300 mg, 350 mg, 450 mg, and 451 mg),

prunus cerasus (“tart cherry”) in amounts of about 10 mg to about 600 mg (e.g., 9.5 mg, 10 mg, 35 mg, 70 mg, 90 mg, 140 mg, 210 mg, 245 mg, 280 mg, 350 mg, 490 mg, 600 mg, and 601 mg),

tanacetum parthenium (“feverfew”) in amounts of about 10 mg to about 300 mg (e.g., 9.5 mg, 10 mg, 17.5 mg, 31.5 mg, 35 mg, 63 mg, 70 mg, 100 mg, 140 mg, 250 mg, 300 mg and 301 mg),

seed of carthamus tinctorius (“safflower seed”) in amounts of about 1 mg to about 300 mg (e.g., 0.9 mg, 3.5 mg, 5 mg, 7 mg, 7.5 mg, 10 mg, 17.5 mg, 35 mg, 52.5 mg, 70 mg, 105 mg, 210 mg, 250 mg, 300 mg and 301 mg),

Magnesium salt (e.g., divalent cation supplied as a sale, e.g., Orotate/Citrate) in amounts of about 10 mg to about 600 mg (e.g., 9.5 mg, 10 mg, 35 mg, 70 mg, 90 mg, 122.5 mg, 140 mg, 175 mg, 210 mg, 245 mg, 350 mg, 420 mg, 600 mg, and 601 mg), and

a hypericum species (“St. John's Wort”) in amounts of about 1 mg to about 300 mg (e.g., 0.9 mg, 3.5 mg, 5 mg, 7 mg, 7.5 mg, 10 mg, 17.5 mg, 35 mg, 52.5 mg, 70 mg, 105 mg, 210 mg, 250 mg, 300 mg and 301 mg).

Material from plants, such as Soybeans, Tart Cherry, Feverfew, Safflower Seed, St. John's Wort, and the like, can be obtained in a variety of forms, for example, fruit, seeds, roots, oil, tincture, leaves, skin or bark. The end material used in compositions in some embodiments of the present invention can be, for example, enclosed in a capsule or tablet, or provided as a liquid, beverage, sublingual, bar, powder, inhalant or topical formulation.

Magnesium is delivered in a salt form from a variety of sources. For example, orotate, citrate and lactate, among others. The end material used in compositions in some embodiments of the present invention can be, for example, enclosed in a capsule or tablet, or provided as a liquid, beverage, sublingual, bar, powder, inhalant or topical formulation.

### Weight Percent of Compositions

Compositions can include varying amounts of PS or SDPS, Tart Cherry, Feverfew, Safflower Seed, St. John's Wort, and/or Magnesium. For example and as shown in Table 1, a composition can be prepared containing the following relative weight percentages of the indicated components, where SDPS is supplied as a lipid powder, tart cherry is supplied as a powder, feverfew is supplied as a powder, Safflower is supplied as powder, and magnesium is supplied as a salt powder or crystal. The relative weight percentages indicated in Table 1 are approximate. For example, a composition including about 30% of SDPS includes compositions in which the relative weight of SDPS is 30%±3% (e.g., Example Composition 1 may include 27% SDPS and 73% Tart Cherry, or 29% SDPS and 71% Tart Cherry, or 32% SDPS and 68% Tart Cherry). Additionally, the composition may further include a blending agent, such as silicon dioxide. The blending agent may comprise about 1%-2% of the total weight of the composition. For example, Example Composition 1 may include 1% silicon dioxide, 29.5% SDPS and 69.5% Tart Cherry. In addition, St. John's Wort can be included in place of, or in addition to, Feverfew and/or Safflower Seed. For example, St. John's Worth can be included in compositions by a relative weight percentage of about 1% to about 30%.

TABLE 1
Example compositions with Ingredients by Relative Weight Percentage

| Example | | PS | Tart | | Safflower |
|---|---|---|---|---|---|
| Composition | Magnesium | (SDPS) | Cherry | Feverfew | Seed |
| 1 | 0 | 30 | 70 | 0 | 0 |
| 2 | 35 | 35 | 30 | 0 | 0 |
| 3 | 20 | 10 | 20 | 20 | 30 |
| 4 | 30 | 20 | 40 | 5 | 5 |
| 5 | 50 | 15 | 10 | 10 | 15 |
| 6 | 60 | 20 | 10 | 9 | 1 |

### Formulations

Embodiments of the present invention can be delivered in a wide variety of forms, including for example, a capsule, tablet, liquid, beverage, sublingual, bar, powder, inhalant or topical.

### Total Weight of Compositions & Daily Intake Protocols

With respect to Table 1, the total additive weight of the enumerated ingredients in the compositions of the indicated embodiments can be for example 100 mg, 250 mg, 250 mg, 500 mg, or 700 mg. Optionally, additional weight may be contributed by additional ingredients, including, without limitation, excipients, stabilizers, solubilizers, preservatives, modifiers, anti-caking agents, flavoring agents, and colorants. For example, in liquid formulations, additional components could include water and flavor (e.g., sweetening agent). In a solid formulation (e.g., a dietary supplement “bar”), additional components could include carbohydrates (complex carbohydrates, simple carbohydrates (sugars), caffeine, ginseng, ginkgo biloba, proteins, amino acids, fats, oils, lipids, probiotics, ions (sodium, potassium, calcium, iron, magnesium), etc.).

In one embodiment, compositions are administered to subjects in a daily amount of about 350 mg per day to about 700 mg per day. For example, in embodiments of the present invention, compositions can be administered as 350 mg capsules. Administration can occur, for example, once, twice, three, or four times a day, and may occur at a specific time, e.g, at approximately 8 AM, 10 AM, noon, 2 PM, 4 PM, 6 PM, 8 PM, 10 PM, midnight, 2 AM, 4 AM or 6 AM of actual time or subjective circadian time.

Compositions can include physiologically beneficial amounts of at least one cortisol reducing agent and/or at least one melatonergic agent. Nonlimiting ranges of physiologically beneficial amounts of particular components of a composition are shown in Table 2. The amounts indicated in Table 2 are approximate. For example, a composition including about 30 mg of SDPS can include 29 mg, 30 mg, or 31 mg of SDPS. Additionally, the amounts listed in Table 2 are recommended amounts for inclusion in, for example, one or two capsules, and reflect daily amounts.

TABLE 2
Recommended beneficial Amounts of
Supplement Components (some embodiments)

| | Recommended | Recommended |
|---|---|---|
| Composition | Physiologically Beneficial | Physiologically Beneficial |
| Element (or | Amount (Recommended | Amount (Recommended |
| component | Minimum Amount) in | Increased Amount) in |
| thereof() | Mg/day | Mg/day |
| Magnesium | 70 | 250 |
| SDPS | 30 | 130 |
| Tart Cherry | 30 | 250 |
| Feverfew | 15 | 100 |
| Safflower Seed | 3 | 110 |

In a particular embodiment, a composition, such as Example Composition 6 (Table 1) includes about 210 mg of Magnesium, about 70 mg of SDPS, about 35 mg of Tart Cherry, about 31.5 mg of Feverfew, and about 3.5 mg of Safflower.

Various Intake Protocols are shown in Table 3, representing the recommended intake amounts of a composition of the present invention, listed by health concern. To address increased or elevated stress, intake may increase by 100 mg, 250 mg, or 350 mg.

TABLE 3
Intake Protocols by Health Concern

| | INTAKE | INTAKE |
|---|---|---|
| | PROTOCOL | PROTOCOL |
| | FACTORS | FACTORS |
| Health | in Mg/Day | in Mg/Day |
| Concern/Aid | (Standard) | (Elevated Stress) |
| Physical Training | 350 | 700 |
| Jet Lag | 350 | 700 |
| Dieting | 350 | 350 |
| Anti-Depression | 350 | 350 |
| Eye Health | 350 | 350 |
| Weight Loss | 350 | 700 |
| Digestion | 350 | 700 |
| Mood | 350 | 700 |
| Depression | 350 | 700 |

### Route of Administration

Routes of administration include oral, nasal or intravenous. Capsules and/or tablets may be taken orally, with or without water. Alternatively, the dietary supplement can be suspended in a liquid, such as water, that may be ingested orally. For example, the dietary supplement can be included in, or provided as a beverage to drink or a spray to be applied to the tongue. Alternatively, the dietary supplement can be included in a bar, such as an energy bar, sports bar, granola bar, cereal bar, or the like, that may be ingested.

### Treatments

In some embodiments, compositions as described herein are administered to a subject in order to achieve one or more of the following goals: regulated circadian rhythms, healthful sleep, resistance to negative effects of ambient light/blue light, inhibition of TDO or IDO, anti-aging, anti-depressive, mood elevation, weight loss, physical training and jet lag.

### EXAMPLE 1

In one example, the composition is a melatonin-protective sleep aid. Inhibiting blue light induced IDO or TDO activation spares melatonin and aids restful sleep.

### EXAMPLE 2

In another example, the composition is supplement providing anti-aging benefits. Poor sleep shortens telomeres, a key marker of aging. Middle-aged and older individuals suffer more sleep disorders with aging. Poor sleep is associated with Alzheimer's and other neurodegenerative diseases.

### EXAMPLE 3

In another example, the composition is a supplement that assists digestion. Irritable Bowel Syndrome is associated with lower tryptophan levels. Protecting tryptophoan from IDO activation-catabolism can raise levels of this amino acids, which can enhance bowel health.

### EXAMPLE 4

In another example, the composition is a supplement that can provide anti-depressive benefits. Inhibiting IDO activation leads to higher levels of tryptophan, sparing this hormone for improved serotonin levels. Low serotonin and tryptophan levels are associated with depression.

### EXAMPLE 5

In another example, the composition is a supplement that can assist with mood disorders. Many psychiatric alterations are strongly associated with TDO and poor sleep. Mood and serotonin levels are linked. Sparing tryptophan assists serotonin levels.

### EXAMPLE 6

In another example, the composition is a supplement to assist with physical training. Sleep promotes attentiveness and performance. Adaptation to workloads increases, resulting in performance and recovery gains. Sufficient sleep aids in fat loss.

### EXAMPLE 7

In another example, the composition is a supplement that promotes weight loss and/or assists with a weight-loss program. Insufficient sleep reduces insulin sensitivity, promoting overeating and obesity. Healthy sleep aids in weight loss programs.

### EXAMPLE 8

In another example, the composition is a supplement to assist with protecting eyes from LED screen exposure. Blue light damages cornea and promotes cataract formation. Anthocyanidins help reduce blue light damage from digital screens.

### EXAMPLE 9

In another example, the composition is a supplement to alleviate jet lag resulting from travel. Resetting internal circadian clock is important in travelers.

### EXAMPLE 10

A composition was created by blending 150 mg of dried organic Tart Cherry (Van Drunen Farms, Momence, Ill.) with 100 mg of SDPS (Chemi Nutra, White Bear Lake, Minn.), with 50 mg of magnesium orotate, with 25 mg of dried Feverfew dried powder (Starwest Botanicals, Sacramento, Calif.), and with 25 mg dried Safflower Seed powder (Starwest Botanicals, Sacramento, Calif.). The composition was dry-blended and encapsulated into vegetarian capsules.

### EXAMPLE 11

A subject was administered the composition of Example 10 daily over a 14-day course. Subjective sleep perceptions were improved: more rapid onset, deeper, more restful was experienced, with ease of waking. Of particular note, next day grogginess was absent. Next day reports included feeling more rested, with improved mood and mental focus. Administration occurred before bedtime at approx. 9:00 PM. One 350 mg capsule was taken with water, daily. Benefits were noted after the first administration and continued throughout the 14-day course days. After a washout period of 15 days, the trial was repeated with similar benefits.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A supplement, comprising:
phosphatidylserine; and
a tart cherry component.

2. The supplement of claim 1, wherein the phosphatidylserine is a plant-derived phosphatidylserine.

3. The supplement of any one of claims 1-2, wherein the plant-derived phosphatidylserine is a soy-derived phosphatidylserine (“SDPS”).

4. The supplement of any one of claims 1-3 wherein the phosphatidylserine constitutes between about 10% and about 35% by mass of the supplement.

5. The supplement of any one of claims 1-4, wherein the supplement includes about 30 mg to about 130 mg of SDPS.

6. The supplement of any one of claims 1-5, wherein the tart cherry component is any one of dried tart cherry, tart cherry powder, and tart cherry extract.

7. The supplement of any one of claims 1-6, wherein the tart cherry component constitutes between about 10% and about 70% by mass of the supplement.

8. The supplement of any one of claims 1-7, wherein the supplement includes about 30 mg to about 250 mg of tart cherry.

9. The supplement of any one of claims 1-8, further comprising divalent magnesium.

10. The supplement of claim 9, wherein the divalent magnesium is supplied as an orotate salt.

11. The supplement of claim 9 or 10, wherein the divalent magnesium constitutes between about 20% and about 60% by mass of the supplement.

12. The supplement of any one of claims 9-11, wherein the supplement includes about 70 mg to about 250 mg of divalent magnesium.

13. The supplement of any one of claims 1-12, further comprising a feverfew component.

14. The supplement of claim 13, wherein the feverfew component constitutes between about 5% and about 20% by mass of the supplement.

15. The supplement of claim 13 or claim 14, wherein the supplement includes about 15 mg to about 100 mg of the feverfew component.

16. The supplement of any one of claims 1-15, further comprising a safflower seed component.

17. The supplement of claim 16, wherein the safflower seed component constitutes between about 1% and about 30% by mass of the supplement.

18. The supplement of claim 14 or claim 15, wherein the supplement includes about 3 mg to about 110 mg of the safflower seed component.

19. The supplement of any one of claims 1-18, further comprising an encapsulant.

20. The supplement of claim 18, wherein the encapsulant is selected from the group consisting of chitosan, cyclodextrin, cyclodextrin dendrimer, lecithin, and a plant protein.

21. A diet supplement, comprising:
plant-derived phosphatidylserine in a physiologically beneficial amount; and
at least one plant-based concentrate or extract which promotes a melatonergic benefit.

22. The diet supplement of claim 21, wherein the plant-derived phosphatidylserine is soy-derived phosphatidylserine.

23. The diet supplement of claim 21 or claim 22, wherein the physiologically beneficial amount of phosphatidylserine is of about 30 mg to about 130 mg

24. The diet supplement of any one of claims 21-23, wherein the at least one plant-based concentrate or extract is of prunus cerasus.

25. The diet supplement of claim 24, wherein the supplement includes about 30 mg to about 250 mg of prunus cerasus.

26. The diet supplement of any one of claims 21-25, wherein the at least one plant-based concentrate or extract is of tanacetum parthenium.

27. The diet supplement of claim 26 wherein the supplement includes about 15 mg to about 100 mg of tanacetum parthenium.

28. The diet supplement of any one of claims 21-27, wherein the at least one plant-based concentrate or extract of carthamus tinctorius.

29. The diet supplement of claim 28, wherein the supplement includes about 3 mg to about 110 mg of carthamus tinctorius.

30. The diet supplement of any one of claims 21-29, wherein the phosphatidylserine and the at least one plant-based concentrate or extract are contained within a capsule.

31. The diet supplement of any one of claims 21-30, wherein the phosphatidylserine and the at least one plant-based concentrate or extract are contained within a tablet.

32. The diet supplement of any one of claims 21-31, wherein the phosphatidylserine and the at least one plant-based concentrate or extract are contained within a liquid for oral administration.

33. The diet supplement of any one of claims 21-32, wherein the phosphatidylserine and the at least one plant-based concentrate or extract are contained within an edible bar.

34. The diet supplement of any one of claims 21-33, wherein the phosphatidylserine and the at least one plant-based concentrate or extract are contained within a powder.

35. The diet supplement of any one of claims 21-34 wherein the phosphatidylserine and the at least one plant-based concentrate or extract are contained within a topical medicament.

36. The diet supplement of any one of claims 21-35, wherein the phosphatidylserine and the at least one plant-based concentrate or extract are contained within a sublingual tablet or strip.

37. A composition, comprising:
about 50% to about 70% of a magnesium component;
about 10% to about 30% soy-derived phosphatidylserine (SDPS);
about 5% to about 20% of a tart cherry component;
about 5% to about 15% of a feverfew component; and
about 1% to about 5% of a safflower seed component.

38. A composition, comprising;
about 150 mg to about 250 mg of a magnesium component;
about 50 mg to about 100 mg soy-derived phosphatidylserine (SDPS);
about 25 mg to about 45 mg of a tart cherry component;
about 25 mg to about 45 mg of a feverfew component; and
about 1 mg to about 5 mg of a safflower seed component.

39. A method of treating a condition in a subject, comprising:
administering the supplement or composition of any one of claims 1-38 to the subject.

40. The method of claim 39 wherein the condition is any one of jet lag, depression, weight loss, mood disorder, dysthymia, anxiety, seasonal affective disorder, sleep disorder, circadian rhythm disorder, light-induced sleep disruption, or a combination thereof.

41. A method of providing a healthful composition to a subject, comprising administering the composition, supplement or diet supplement of any of claims 1-38 to a subject.

42. The method of claim 41, wherein the healthful composition is provided during at least one of physical training and dieting.

43. A method of promoting wellbeing in a subject, comprising:
providing the composition, supplement or diet supplement of any of claims 1-38; and
administering said composition, supplement, or diet supplement to the subject.

44. The method of claim 43 wherein the wellbeing being promoted is wellbeing of at least one of eye health and digestion.

45. A method of regulating activity of at least one of tryptophan 1,3-dioxygenase (TDO) and indoleamine 2,3-dioxygenase 1 (IDO) in a subject, comprising:
administering the supplement or composition of any one of claims 1-38 to a subject.

46. A method of regulating cortisol in a subjecting, comprising:
administering the supplement or composition of any one of claims 1-38 to a subject.
